(19) United States
(12) Patent Application Publication
(10) Pub. No.: US 2018/0147346 A1
(43) Pub. Date: May 31, 2018

(54) INFUSION SYSTEM AND METHOD FOR INTEGRITY MONITORING OF AN INFUSION SYSTEM

(71) Applicants: David Grosse-Wentrup, Münster (DE); Uvo Hölscher, Steinfurt (DE)

(72) Inventors: David Grosse-Wentrup, Münster (DE); Uvo Hölscher, Steinfurt (DE)

(21) Appl. No.: 15/597,698

(22) Filed: May 17, 2017

Related U.S. Application Data
(63) Continuation of application No. 15/300,295, filed on Nov. 28, 2016.

(51) Int. Cl. A61M 5/168; A61M 5/142; A61M 5/36; A61M 5/14

(52) U.S. Cl. CPC A61M 5/16859; A61M 5/1408; A61M 5/14228; A61M 5/14232; A61M 5/16886; A61M 5/365; A61M 2205/14; A61M 2205/15; A61M 2205/17; A61M 2205/18; A61M 2205/3306; A61M 2205/3334; A61M 2205/3372; A61M 2205/3375; A61M 2205/6081; A61M 2205/6018; A61M 2205/6054; A61M 2205/70

(57) ABSTRACT

An infusion system for transporting fluid into a patient and a method for integrity monitoring of the system. The infusion system includes one or more sources of fluids to be infused, such as pumps or reservoirs, and a plurality of hose lines that carry the fluid to an entry point at the patient. A signal generator introduces a pressure signal into the fluid and a sensor, spaced some distance from the signal generator, receives the sensor signal and generates an input signal based on the sensor signal and transmits the input signal to an evaluation circuit. Information transmitted to the evaluation circuit is displayed visually, the information relating to operating status of fluid sources and lines, as well as to flow rates, blockages, leaks, etc.

### BACKGROUND INFORMATION

### Field of the Invention

The invention relates to an infusion system, as well as a method for monitoring the integrity of an infusion system.

### Discussion of the Prior Art

Conventional infusion systems are known in the field of medical care. They are used to infuse fluids into a patient, for example, into the stomach or into a blood vessel of the patient. All infusion systems have an infusion source and an infusion tube line that is connected at its distal end (farthest from the patient) to this infusion source and at its proximal end (closest to the patient) to an opening or patient access point, for example, to a stomach tube, a venous catheter, etc. The term tube line is used hereinafter, when reference is to one or more specific tube lines and the term tubing used when reference is to the tubes in general or the material used for the tubes. Fluid to be infused is transported by a flow device that initiates forward flow of the fluid from the infusion source and through the tubing to the patient access point, where the fluid then exits the infusion system and is infused into the patient. The flow device can be apparatus that takes advantage of force of gravity or an infusion pump.

Elements of the infusion system are regularly replaced for hygienic reasons, for example, every 24 hours, in order to avoid microbial growth or contamination. The replacement is done manually and errors can occur. For example, the tubing may be connected incorrectly, with the consequence that leaks occur in the infusion system. Stenosis can occur as a result of the patient moving, i.e., a tube line has a kink in it and the intended flow of the fluid to the patient access point is restricted or completely interrupted. Also, the specified settings may inadvertently be improperly or incompletely adjusted on any one of various components in the system, such as stopcocks, multi-port valves, or similar devices, either due to actions or movement by the patient or due to incorrect setup of the infusion system.

Typically, conventional infusion systems encompass not just a single infusion source with a single tube line. Rather, it is common practice to administer multiple medications to a patient simultaneously; thus, often two, three, or more, sometimes as many as six medications are prescribed for a patient, whereby a dedicated infusion source is provided for each medication. The tube lines that are connected to the sources typically feed into a common tube line within the infusion system, for example, by means of T or Y connectors, with the common tube line then feeding into the patient access point, for example, into a vein cannula. It is important with such multi-medication infusion systems that the individual medications are fed from the individual infusion sources into the common tube line at certain locations or at certain times, because medications that are in contact with each other for an extended period of time may interact with each other in a way that results in a reduction in the efficacy of the medications or a malfunction of the infusion system. For example, a prolonged combination of medications could possibly result in flocculation or precipitating out of one or more of the medications inside the common tubing and possibly block the patient access point or a filter upstream of the access point. Thus, it is desirable to keep the time and distance when multiple medications are being transported together through the common tube line to a minimum.

It is often difficult for medical personnel to visually monitor the proper functioning of the infusion system, particularly in an intensive care unit or an operating room. In these situations, most of the patient's body is often covered or draped with a sheet or blanket, and because of that, the layout of the individual tube lines and any branching points in the infusion system are frequently covered up, so that it is not possible for the medical personnel to adequately monitor the infusion system.

An increase in pressure in a tube line is an indication that there is an occlusion somewhere in the system. For example, a kink in a tube line, i.e., a stenosis, may restrict or completely block the flow of the fluid, which leads to the increase in pressure. For that reason, the conventional infusion system typically has some means of detecting a pressure build-up in the system. One method is to provide a pressure sensor that detects pressure build-up. Another method is to monitor energy consumption. The energy consumption of a pump that is working properly is a known value. If there is increased pressure in the system, then the pump has to work harder against the increased pressure and, as a result, the energy consumption increases.

These methods of monitoring the infusion system are “passive” methods, in that they do not actively monitor the proper functioning of the system, but merely detect a build-up of pressure after something has gone wrong. This type of monitoring is inadequate, however, when the flow rates of the fluids to be infused are very low, which is the case when high-potency medications are being infused. These medications are typically administered in very low dosages per unit of time. As a result, an increase in pressure due to a blockage occurs only very gradually and may not reach a measurable or detectable value for an extended period of time. Hence, a notification or alarm may not occur until long after the blockage has occurred.

Leaks can also occur in the infusion system, for example, at places where a section of tubing is connected to another component in the system, such as to an infusion source, to a branching connector, filter, stopcock, multi-port valve, or to the patient access point, or when the access point itself becomes detached from the body of the patient. Monitoring an increase in pressure or energy consumption does not detect these leaks, however, because there is no increase in pressure.

What is needed, therefore, is an infusion system and a method that actively and automatically monitors the functional integrity of the infusion system. What is further needed is such a method that automatically recognizes the various components of the system.

### BRIEF SUMMARY OF THE INVENTION

It is an object of the invention to provide an infusion system and a method of monitoring the infusion system that not only monitors the flow of the fluid to be infused, but also monitors the configuration of the system and the operating states of various components in the system. It is a further object of the invention to actively monitor the functional integrity of the infusion system.

The infusion system according to the invention includes the conventional components of an infusion system, such as a source for the infusion fluid, a flow device, infusion tubing for carrying the fluid to a patient access point, and some means of forcing the fluid to flow to the patient access point. In addition, the infusion system includes components that actively detect the flow rate of the fluid and also the various components in the system and their operating states. These components provide a method of automatically and continuously monitoring of the integrity of the system.

Infusion systems are frequently used to administer more than one fluid simultaneously to a patient and for that reason, the discussion hereinafter will describe an infusion system that has more than one infusion source, each infusion source having its own tube line feeding from the source to a patient access point, and, for each tube line, a signal generator and a sensor. It is understood, however, that this description excludes an infusion system according to the invention that has a single infusion source and tube line.

Preferably, a signal generator and a sensor is provided for each tube line in the infusion system, whereby the sensor is placed some distance away from its respective signal generator whereby the signal generator is typically placed at or near the beginning of the tube line and the sensor at the end of the line. The signal generator actively introduces a signal into the fluid and the sensor detects the signal. The signal may be provided as a single pulse or a series of pulses or as a longer continuous signal. The signal generated by the signal generator is influenced by conditions in the infusion system as it travels through the fluid to the sensor and is, therefore, changed in some way by the time it reaches the sensor.

The sensor signal is then transmitted by the sensor either directly or indirectly, i.e., after signal processing, to an evaluation circuit that is operatively connected to the sensor and that processes and evaluates the signal received from the sensor. The signal generated by the signal generator is referred to hereinafter as a sensor signal and the signal forwarded by the sensor to the evaluation circuit as an input signal. An evaluation of the information the sensor signal is carrying and the changes that occur in the course of its flow path enables conclusions to be drawn regarding the state of the infusion system.

A display device is operatively connected to the evaluation circuit, and a graphic representation of the information that is derived from the analysis in the evaluation circuit is shown on the display device. The display may be in the form of a diagram or a “system map”, similar to the types of diagrams or maps that are used to show the various train or streetcar lines in a public transportation system. Ideally, this system map displays the individual components of the infusion system, including all of the tube lines. The map may also indicate the flow rate of the fluid, for example, by using color-coding to indicate flow rate is within a specified range or deviates from the specified range, or by providing an animate display showing moving symbols that indicate the prevailing flow rate in a particular tube line. The display may also indicate that the system is in complete working order and, as applicable, provide information on a detected error.

Various types of sensor signals are suitable to provide the desired information and, accordingly, various types of signal generators may be suitable. For example, the signal generator may be constructed as a pressure generator that sends pressure signals into the fluid or may be a sound generator that sends a pulse that is a sound wave into the fluid, or may be a light generator that generates light signals.

For purposes of illustration, the flow device is referred to hereinafter as an infusion pump, although other types of devices or equipment may be used to initiate flow. The signal generator may be provided directly in the infusion pump, which is an advantage, because medical personnel do not have to deal with an additional device. This degree of integration also has an advantage when the signal generator is constructed as a pressure generator and the infusion pump is an injection pump, because the infusion pump may be provided with a control that enables a micro-modulation of the pump rate. Thus, for example, the plunger motion on the injection pump generates a pressure pulse.

Alternatively, the signal generator may be connected to the infusion tubing, instead of to the pump. This configuration of the infusion system allows the continued use of already existing conventional infusion pumps, because the signal generator is able to be retrofitted into the system without requiring any modification of the pump. In this case, the signal generator is provided as an intermediate element that is inserted into the infusion tube line, such that the fluid flows through the signal generator. This ensures the most direct contact of the signal generator with the fluid, i.e., the wall of the tubing is not between the fluid and the signal generator. This is an advantage, because the different materials used for tubing could possibly lead to a change in the results or require regular re-calibration of the infusion system, whenever the tubing is changed.

As mentioned above, the infusion system may encompass multiple infusion sources and, thus, also multiple infusion tube lines and a corresponding number of signal generators. Ideally, an individual signal generator is provided for each of the different infusion fluids, with each signal generator generating a unique pulse or signal. Thus, a unique sensor signal is generated for each fluid, so that each individual measurement value and sensor signal are clearly identifiable as relating to a particular one of the fluids. This provides particularly clear, precise and far-ranging information as to the state of the infusion system.

Rather than providing a plurality of signal generators, one for each infusion fluid, it is possible to provide a single signal generator at a confluence of tube lines, i.e., where a plurality of tube lines converge into a common feed line. This keeps the equipment requirements of the infusion system to a minimum. It is possible to obtain precise monitoring and differentiated information about the individual components of the infusion system, particularly also about the individual segments of the tube lines, by providing an individual signal receiver for each infusion source, i.e., for each of the different fluids.

The sensor may be constructed as an actuator sensor element, so that the sensor not only receives the pulsed signals and sends out input signals for the evaluation circuit, but is also able to generate pulses. The use of two actuator/sensor elements placed a distance apart from each other makes bi-directional data transmission possible. Signals are emitted in two different directions within the fluid, and the flow velocity of the fluid is determined automatically, based on the differences in travel time of the corresponding sensor signals.

Different materials may influence for the sensor signals or the forward transmission of the signals in different ways. For that reason, it is advantageous to emit the sensor signal in the form of a modulated signal, for example, with different frequencies. Thus, different tubing materials that attenuate certain frequencies more than others do not negatively influence the forward transmission of the pulse signal, because those frequencies on the modulated signal that are not attenuated by the particular tubing material are transmitted to the sensor with sufficiently strong signal strength.

The same applies for other components that are provided within the infusion system, for example, filters, valves, stopcocks, branching connectors, etc., which, depending on the material and also depending on the settings of the stopcocks and multi-port valves, can present an obstacle for the transmission of the pulse signal. Transmitting modulated signals significantly increases the probability that at least one part of the signal is able to pass through the corresponding components of the infusion system and reach the sensor with sufficient signal strength.

In addition, conclusions may be drawn automatically as to the state of the infusion system or its individual components, based on which portions of the signal are weakened or suppressed and which portions of the signal reach the sensor with a significantly greater signal strength, and the appropriate information may be transmitted to the evaluation circuit and then presented on the display.

Careful signal analysis also makes it possible to detect the presence of gas bubbles, for example, air bubbles, in the fluid-filled tube lines. The size of these gas bubbles may also also be determined in this way.

In summary, the method according to the invention actively introduces one or more sensor signals into the fluid to be transfused. The sensor signal may be carrying general information, such as the type and concentration of medication in the fluid, the specified flow rate, or information relating to a component in the system, such as the pump ID. Changes occur to the sensor signal as it travels through the fluid. The sensor signal is received in a sensor that then forwards an input signal to the evaluation circuit. The input signal may be a processed signal derived from the sensor signal or be identical to the sensor signal as it is received at the sensor. The evaluation circuit, a component that is operatively connected to the sensor, analyzes the input signals for the information it is carrying and, based on equations, also determines from the changes that occurred in the sensor signal as it travelled through the fluid, what the flow rate of the fluid is. The information gleaned in the evaluation circuit is then graphically presented in a system map that is displayed on a monitor.

The method according to the invention provides a way of monitoring the integrity of the overall infusion system, not just errors. The method reduces the sources of error, and provides assurance to the medical personnel that the system is functioning properly.

The method monitors the state of the fluid system, i.e., the infusion tube lines and all components that are connected to them, from the time the system is set up and throughout the entire operating time, and it does this automatically. The configuration of the entire system is monitored, so that incorrect setup of the system is immediately recognized and some form of notice given, thereby preventing incorrect operation.

The integrity monitoring is based on analysis of the transmission behavior of signals transmitted through the fluid. This analysis provides information on the individual system components, such as pumps, tubes, valves, patient access points, etc., the relevant characteristics of the components, such as length of tube, valve settings, flow rates, etc., and the locations of the components in the system and their operating states (on/off, active/inactive, open/closed). Available electronic or machine-readable data is also incorporated into the information that is processed in the evaluation circuit, so that the integrity monitoring method according to the invention is able to perform an automatic check for completeness, correctness, compatibility, and safety of the infusion system.

The information gathered and analyzed in the evaluation circuit is then displayed in a schematic image, i.e., a system map, of the entire infusion system. In other words, the method presents a comprehensive image of the entire infusion system, based on the detected information, without negatively affecting the functioning of the system. This knowledge about the system serves the immediate recognition of errors in the setup, function, wiring, and connections, prevents harm to patients, and provides assurance to medical personnel, that the infusion system is functioning properly.

### BRIEF DESCRIPTION OF THE DRAWINGS

The present invention is described with reference to the accompanying drawings. In the drawings, like reference numbers indicate identical or functionally similar elements.

FIG. 1 is an example of a system map that provides a schematic illustration of the infusion system according to the invention.

FIG. 2 is a schematic illustration of a second embodiment of the infusion system, illustrating the placement of signal generators and sensors.

FIG. 3 shows three different states of an infusion system, showing signal transmission paths during a measuring procedure

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

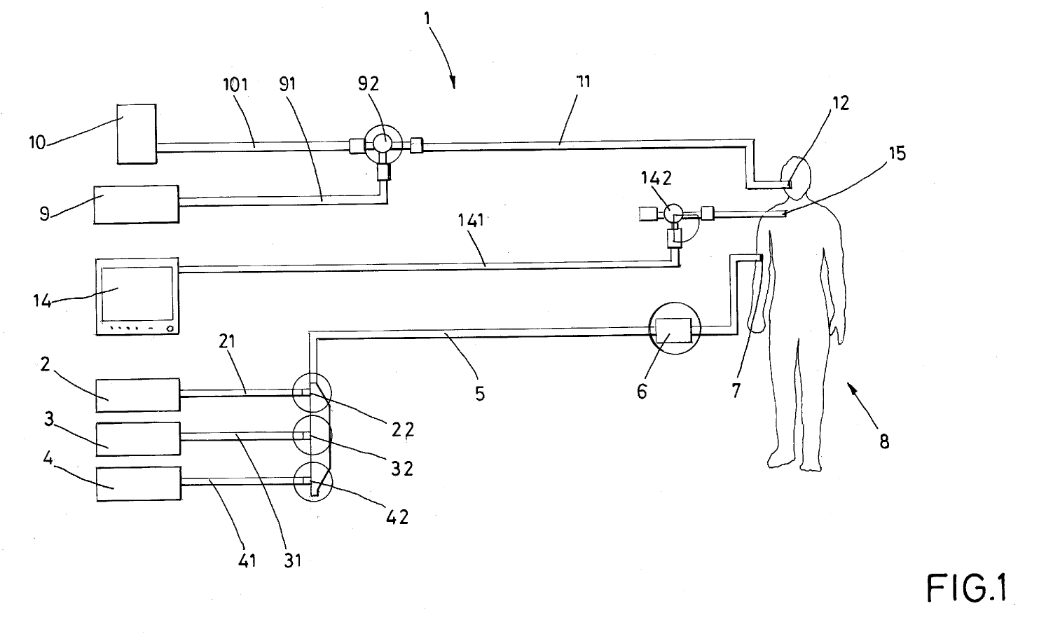

FIG. 1 is a schematic illustration, i.e., a “system map” of an embodiment of an infusion system 1 according to the invention. The basic infusion system 1 comprises at least one infusion pump 2, at least one infusion tube line 21, and at least one multi-port valve 22. The infusion pump 2 forces a medication-carrying fluid that is to be infused into a patient 8 through the infusion tube line 21 to a patient access point 7 at a specified flow rate.

It is very common that multiple medications are administered to a patient, either in combination or separately. And, although it is possible that an infusion system according to the invention would have a single infusion source and single line to a patient access point, the discussion below refers to a system having multiple infusion lines, because this is a much more common setup. In such cases, the infusion pump 2 and the corresponding infusion tube line 21 are provided for each medication. In the embodiment shown in FIG. 1, the infusion system 1 is a complex system that comprises three infusion pumps 2, 3, and 4, and a corresponding number of infusion tube lines 21, 31, and 41 and multi-port valves 22, 32, and 42. The infusion tube lines 21, 31, and 41 feed into a common infusion tube line 5, which runs through a filter 6 and then to a first patient access point 7, which is a venous cannula, i.e., vein tube, that has been inserted into a vein in the arm of a patient 8.

The possible switch settings for the multi-port valves 22, 32, and 42 are “open/permeable in all directions,” “open/permeable in one direction,” or “closed/impermeable in all directions.” Preferably, the multi-port valves 22, 32, and 42 carry some visual identification of the particular switch setting, such as, for example, color-coded rings, green indicating an open setting and red a closed setting, or diagrams indicating a flow-through or a blocked flow state.

In addition to medication-carrying fluids, often an additive solution, for example, a saline solution, and/or a nutrition-carrying fluid is administered to the patient. FIG. 1 shows an additional infusion pump 9, an infusion tube line 91 that carries a saline solution, a metering pump 10, and an infusion tube line 101 that carries a nutritional fluid. The lines 91 and 101 feed into a multi-port valve 92, which guides the fluids into a common infusion tube line 11 that leads to a patient access point 12. In this case, the patient access point 12 is a stomach probe that is guided through the patient's mouth and throat into the stomach.

In addition to infusing fluids into a patient, the heart activity of a patient is frequently monitored. FIG. 1 also shows a patient monitor 14 that is connected via a tube line 141 to a multi-port valve 142 and then to a third patient access point 15, which is a venous catheter. The monitor 14 monitors the heart activity of the patient 8 and displays relevant information.

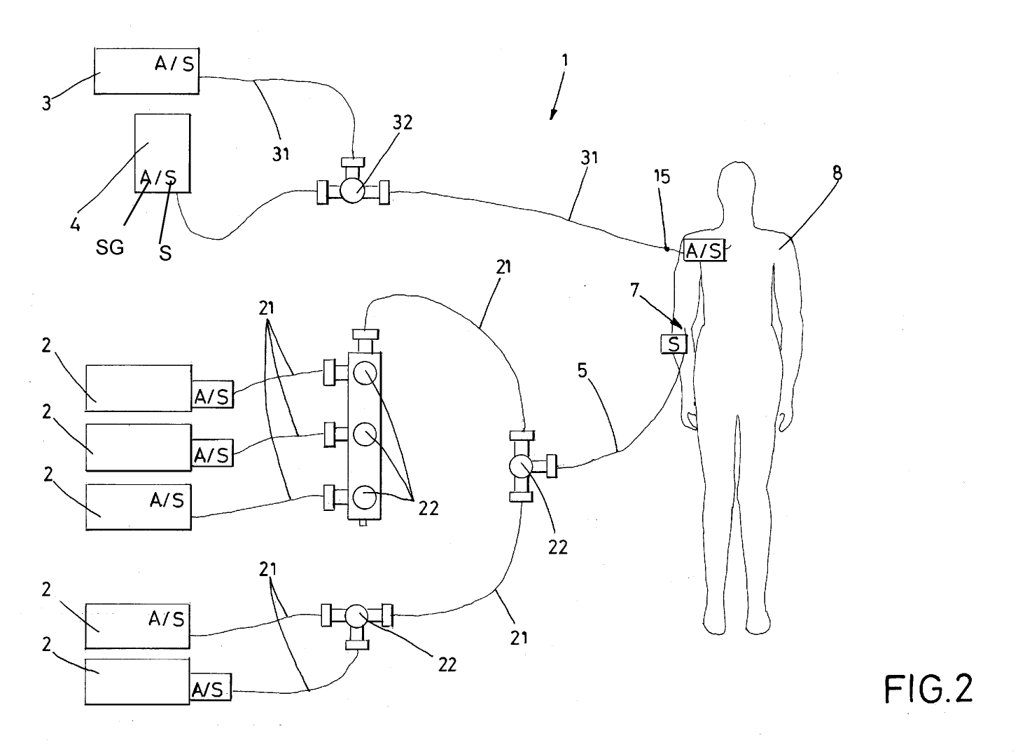

FIG. 2 shows a second embodiment of the infusion system 1 according to the invention, and particularly illustrates components that enable an active monitoring of the state of the infusion system. This infusion system 1 is also a complex system, comprising a plurality of first infusion pumps 2, a corresponding number of tube lines 21, and multi-port valves 22 that feed the fluid into a common tube line 5. The tube line 5 is a trunk line that carries all of the fluids from the plurality of infusion pumps 2 to the patient access point 7, which, in this instance, is a venous catheter.

In the embodiments shown in FIGS. 1 and 2, the system 1 includes devices that enable identification of the components and active monitoring of the functioning of the system, i.e., monitors flow rates, although these devices are not shown in FIG. 1. The devices include one or more signal generators SG that generate a signal that is transmitted through the fluid to be infused, and one or more sensors S that detect the signal emitted by the signal generator. The signal may be a single pulse, a series of pulses, or a longer, continuous signal.

In the preferred embodiment shown in FIG. 2, a plurality of actuator/sensor elements A/S are used, this element including both the signal generator SG and the sensor S. A sensor S is placed at the patient access point 7 and an actuator/sensor element A/S is provided at each of the infusion pumps 2. A second infusion pump 3 is connected to a tube line 31, which feeds through a multi-port valve 32 and on to the patient access point 15, a venous catheter. An actuator/sensor element A/S is provided at the pump 3 and at a patient access point 15 to enable a bi-directional transmission of sensor signals in the tube line 31. The fluid in the tube line 31 flows in one direction only, from the pump 3 to the patient access point 15. Differences in travel time of the emitted sensor signals arise between the two actuator/sensor elements A/S and these differences are used to determine the flow rate of the fluid being infused.

A third infusion pump 4 has a tube line 41 that feeds into the multi-port valve 32. Fluid then flows into the tube line 31 and on to the patient access point 15. The first and second infusion pumps 2 and 3 shown in FIG. 2 are constructed as injection pumps, each with a syringe plunger that pushes the fluid into the respective tube lines 21 and 31. The third infusion pump 4, on the other hand, is constructed as a peristaltic pump, just as an example, to demonstrate that different types of pumps may be used within the same infusion system 1.

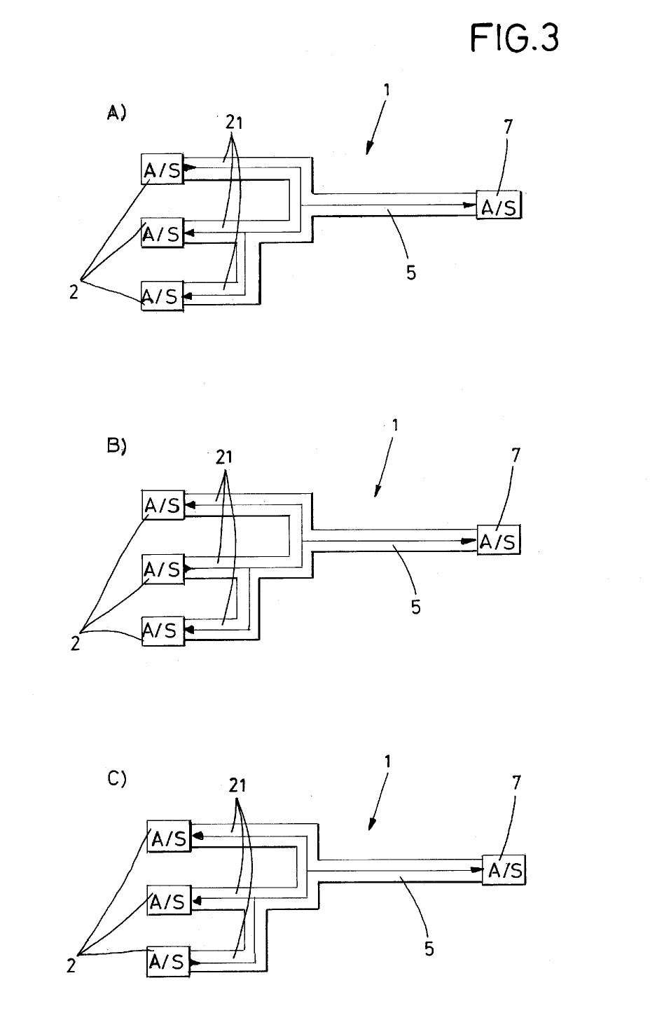

FIG. 3 illustrates three different operating states A, B, and C of a third embodiment of an infusion system 1 comprising three infusion pumps 2. Each of the three infusion pumps 2 has an infusion tube line 21 that feeds at some point downstream into the common infusion tube line 5, which then feeds into the patient access point 7. An actuator/sensor element A/S is provided at each of the pumps 2, as well as at the patient access point 7. Directional arrows on the actuator/sensor elements A/S indicate the direction in which a signal is sent out and/or travels through the fluid.

In state A, the signal generator in the actuator/sensor element A/S of the upper infusion pump 2 emits a signal that travels through the fluid in the tube lines 21 and 5 to the actuator/sensor elements A/S of the other infusion pumps 2 and to the patient access point 7 and is detected by the sensors in those actuator/sensor elements.

In state B, the actuator in the actuator/sensor element A/S of the middle infusion pump 2 emits a signal that travels to the actuator/sensor elements A/S of the other infusion pumps 2 and the patient access point 7 and is detected by the sensors in those actuator/sensor elements.

In state C, the actuator in the actuator/sensor element A/S of the lower infusion pump 2 emits a signal that travels to the actuator/sensor elements A/S of the other infusion pumps 2 and to the patient access point 7 and is detected by the sensors in those actuator/sensor elements.

As shown in FIG. 3, an actuator/sensor element A/S is also provided at the patient access point 7. Thus, the infusion system 1 may be in a state that is not shown in FIG. 3, a state in which the actuator in the actuator/sensor element A/S of the patient access point 7 emits a pulse that travels to the sensors in the actuator/sensor elements A/S of the infusion pumps 2 and is detected by those sensors. The fact that the actuator/sensor element A/S at the patient access point 7 is able to send out a signal that travels in a direction opposite the flow of the fluids to be infused makes it possible to analyze a bi-directional signal transmission as a way of determining the flow rates of the fluids.

The method according to the invention provides an identification of the various components of the system and their interconnections within the system. In the case of wireless components, for example, the sensor signals may be modulated and information relating to a component imprinted on them. Or, control devices may be provided at two locations, for example, one being the actuator/sensor element and the other being a centrally located control unit that also contains the evaluation circuit, and by simultaneously actuating the two devices, for example, by simultaneously pressing a key on each of the devices.

The following information is gathered in the evaluation circuit, which is preferably an electronic evaluation circuit:

Information on the Operating States of the Components in the Infusion System:

Information on the operating states is obtained from the signal evaluation. The transmission behavior of the infusion system is influenced by the components in the system. Different components of the infusion system, such as the tube lines, the materials used for the tube lines, stopcocks, multi-port valves, filters, branching connectors, patient access points, etc., all of these result in characteristic changes in the transmission behavior because the emitted signals produce characteristic echoes, due to absorption, transmission, attenuation and/or reflection. When these characteristics are known, then an analysis of the change in the signal between its emitted state and received state provides information about the type of component (for example, branching piece, filter, etc.), its location within the system, and its setting (open, closed).

Information from the Infusion Pumps:

The packaging for the fluid to be infused typically has a machine-readable code printed on it, for example, an RFID tag, a bar code, or a QR code, that identifies the type of fluid and its concentration. A scanner for the particular type of code may be provided in the infusion source, for example, in the infusion pump, and the information on the packaging then be automatically sent to the evaluation circuit.

Physician Prescription Information:

Healthcare facilities are obligated to maintain a record of any medication that is prescribed. This information is typically stored in the form of electronic data in a data storage unit of the facility, and thus, may be automatically sent to and processed in the evaluation circuit of the infusion system.

Information from Pharmaceutical Data Banks Relating to the Compatibility of Medications:

This information is also typically stored in the form of electronic data in a data storage device of the healthcare facility and may thus also be automatically sent to and processed in the evaluation circuit of the infusion system.

The information gathered by the evaluation circuit is then processed by wirelessly connected hardware/software and a graphic presentation of the information is generated as needed, or when alarms/errors occur, and provided as filtered data to medical personnel.

Three different technologies may be used in the method according to the invention for the physical monitoring of the system, either alone or in combination: acoustical, electrical, and optical. The types and qualities of information the technologies provide complement each other, but they may also be used singly.

Acoustic Monitoring:

In an acoustic method (sound wave results in a change in pressure), longer acoustic signals and/or acoustic pulses and their accompanying pressure surges are introduced into the fluid in the system of tube lines or into the tube material itself. Information that is detectable by the receiving sensor and that identifies the sender/actuator may be imprinted onto the acoustic signal. Disturbances in the fluid propagate as pressure waves, with a pressure wave velocity that is typical for the constellation and the medium. The propagation velocity of the pressure wave is calculated as follows:

$$α = \frac{{α}_{0}}{\sqrt{1 + \frac{{E}_{F}}{{E}_{S}} + \frac{d}{s}}} \qquad Equation 2$$

Pressure Propagation Velocity.

α₀=Pressure propagation velocity
E_{F}=Elasticity module of the fluid
ρ=Density of the fluid

These pressure waves propagate in the tubes with the following velocities:

$${α}_{0} = \frac{{E}_{F}}{ρ} \qquad Equation 1$$

α=Propagation velocity of the pressure wave in the tube
α₀=Velocity of sound in the fluid
E_{F}=Elasticity module of the fluid
E_{R}=Elasticity module of the tube
d=Clear diameter of the tube
s=wall thickness of the tube

The Poisson's ratio of the raw material p goes into the equation as follows:

### Equation 3

### Pressure Wave Velocity in the Tube Including Lateral Contraction

α=αᵢ0/√(1+(E_{ι}F*(1−μ^{⬆}2)/E_{⬇}+R+d/s)

This calculation is required, if highly precise results are required.

Suitable acoustic signals include monofrequency signals (for example, sinus waves), pulses, and multi-frequency sweeps and chirps. Multi-frequency signals are particularly suitable in systems in which frequency dependencies aid in characterizing system properties.

The components used in the system, their properties, the system itself, including its interconnections, are determined by measuring and evaluating the acoustic sensor signals that are introduced into the flow path of the fluid. Fluctuations in pressure generated by the infusion pump or that stem from other sources (for example, the patient) may also be evaluated for this purpose.

The sensor signals are introduced, for example, via their own sound generators that may be connected directly to the tube line or be integrated into the infusion pumps. It is also possible to generate the signals by means of micro-modulation of the flow rates of the infusion pumps. Micro-modulation is understood here to mean the short-term change in the rate of infusion, whereby these changes are significantly shorter in duration than the pharmacological half-times of the fastest medications, in order to exclude changes in the pharmacological efficacy of the infusion. The micro-modulation is characterized in that the net infusion rate does not change over a longer period of time, i.e., decreases in the infusion rate are compensated by subsequent increases.

The signal generators may be integrated into the tubing system by means of intermediate pieces, i.e., connectors. If the signal generators are integrated into the infusion pumps, then the signal-generating elements are incorporated into the pump mechanics. For example, an actuator element is added to the propulsion mechanics of an injection pump, and the syringe that is in the injection pump be used to introduce the sensor signals into the system. The motor driving the infusion pump may also be controlled in a modulated way, so that the corresponding fluctuations in pressure are generated in the tubing system. With peristaltic pumps, an additional peristaltic element placed at the proximal end of the infusion system, i.e., close to the patient, may generate these signals or a generator may generate signals in the fluid by transmitting them through the tubing.

The acoustic sensor signals propagate through the system of tubes as pressure waves and are detected by means of electrical actuator/sensor elements at other points in the system, for example, at intersections or end points. With volumetric pumps, a modified pressure sensor serves to detect the signals.

The characteristics of the signals may differ from each other, depending on the characteristics of the system: Each actuator may, based on its location (for example, in a pump, stopcock, valve, or catheter) have a unique signal characteristic that is coordinated with other components. Additionally, special subsegments of the signal may be used in order to transmit information, such as flow rate setting, medication, pump ID, etc., from one pump to other pumps or to a common receiver via the acoustic system.

Typical algorithms for handling transmission conflicts, such as the Carrier Sense Multiple Access/Collision Avoidance or Carrier Sense Multiple Access/Collision Detection, may be used, to avoid disturbances or errors due to an interaction or overlap of the transmissions. The actuators may, for example, coordinate the timing of signal output when the system is initiated, automatically as part of the self-recognition process. The pumps may be synchronized and halted for a brief time, as needed, as soon as one pump sends out a signal; in other words, the pumps provide time slots for sending and receiving among themselves, in which each one actuator sends out a signal and the other actuator/sensor elements listen for the signal response.

The signal response of the transmitted signals is taken up at multiple or at all other detection points by the respective actuator/sensor elements. The time difference of the oncoming signals is measured and the difference in signal travel time determined as follows:

### Equation 4

### Signal Travel Time Difference

Δt_{S}=t_{S1}−t_{S2}

Δt_{S}=travel time difference [ms]
t_{S1}=time of signal detection at Point 1
t_{S2}=time of signal detection at Point 2

The measured and in part weak signals are processed, using signal processing methods. Lock-in amplification may be used for weak signals. In order to obtain an exact measurement of the travel time, the pressure signals have to be analyzed by means of foot-to-foot algorithms (foot-to-foot radius), peak and edge detection, methods of the smallest squares, as well as auto-correlation and cross-correlation. This is necessary in order to obtain the most exact determination possible of the travel time and also, because the pressure signals themselves change in the course of their travel through the line. The above-mentioned methods may be applied simultaneously, in order to obtain even greater accuracy.

The signal parameters that are relevant here are the travel time (this includes the total travel time of the signal through the system, the ratios of the travel times in the individual paths of the system), and also the change in the wave form (this includes among others amplitude, frequency, and change over time of the wave form or the period), between each of the individual measuring points or in the echoes.

Also, each actuator/sensor element may receive the different echoes of its own output signal. The combination of the total and partial travel times of the signals in the system enables linear systems of equations to be set up to calculate the ratios of the individual lengths of the tubes, using Gaussian elimination methods. The result is a definitive diagram of the interconnections of the partial paths. This diagram contains the individual length ratios, interconnections, branchings, and valve settings of connected elements, as well as an estimation of the absolute lengths. The signal pulse is reflected at the occlusions, for example, at closed stopcocks or at stenoses or blockages.

This reflection or echo is recognized by the transmitting actuator/sensor element and the distance to the occlusion is determined by means of the signal travel time. Also, at such points of occlusion, depending on the material to be penetrated, frequency ranges may be used for the signal that enable the signal to penetrate the particular material more readily. Characteristic absorption and transmission of frequency-modulated signals allow in this way statements to be made as to the position and type (for example, stopcock, T-connector, filter) of the occlusion.

If additional properties (for example, E-modulus, inner and outer diameters) of the components used are known, then the actual lengths of the partial paths may be calculated as follows, drawing on Equations 2 and 4:

### Equation 5

### Length of Partial Paths

l=Δt_{S}*α

l=length of partial path [m]
Δt_{S}=difference in travel time
α=propagation velocity of pressure wave in the line

Even with completely unknown systems, and this may include improperly setup systems with medically irrelevant interconnections, this calculation may be used to determine the position of intersections and end points relative each other, based on the travel times, or by means of metric multi-dimensional scaling.

Furthermore, the previously mentioned sweeps and chirps may be used on unknown systems to map the system by means of the system response.

Infusion tubing with generally unknown modulus of elasticity may be used in the system and characterized by means of a one-time measurement and the following equation, derived from Equation 2:

$${E}_{R} = \frac{1}{[ {( ( {α}_{0} * Δ t ) ]}^{2} - 1 ) * ( \frac{s}{d * {E}_{F}} )} \qquad Equation 6$$

### E-Modulus

E_{R}=E-Modulus of the line [MPa]
α=Velocity of pressure wave propagation in the line
E_{f}=E-Modulus of the fluid
d=Clear diameter of the tube
s=Wall thickness of the tube
Δt=Difference in travel tube

It is also possible to determine the flow rate, based on the velocity of the pressure wave that is changed by the flow and measured by means of bi-directional measurement in the system. In this case, a sensor signal, i.e., a pressure pulse, is sent back and forth between each of two communicating actuator/sensor elements. The flow between the elements is determined from the difference in the travel time as follows:

$$\frac{( \frac{1}{2} Δ t * α ) * A}{\frac{1}{2} Δ t} = F \\ F = Fluss [ \frac{ml}{s} ] \qquad Equation 7$$

Determination of flow by means of bi-directional measurement

A=Surface of the line
Δt=Difference in the signal travel times
α=Velocity of pressure wave propagation in the line

In order to refine and verify the measurement, this method may be supplemented with a Doppler frequency measurement of sinus wave signals, whereby an actuator/sensor element sends out a periodic signal that is detected by the other elements.

Subsequently, the partial flows of the individual sections, as well as the total flow rate of the system may be calculated the same way as before by means of Gaussian elimination methods. The flow rates may then be reconciled with the interconnections diagram and the specified conditions from the fluid management system.

Given a known network and known flow rates in the partial sections, stenoses and leaks may be recognized early, even with low flow rates.

By measuring the transmission behavior of the individual components, the whole system may later be simulated and its properties and function predicted. The entire transmission behavior may then be measured during operation and reconciled with the measured signal travel time. The transmission behavior, as well as the travel time of the signals, depends on the components used, for example, on the tubes and their properties. Thus, components that are alien to the system may be detected by means of the discrepancy of calculated and measured values for travel time and transmission behavior and and this information used to check the reliability of the components for use in the system.

The entire system may be simulated later and its properties and function predicted by measuring the transmission behavior of the individual components.

If systems are constructed from known elements, then additional statements regarding the system may be made, based on the transmission behavior. This applies to the detection of air bubbles, but also to statements regarding the fluids used, specifically, their density and viscosity. Thus, in the case of infusions, this is an additional verification that the proper medication is being administered.

Furthermore, with known systems, the system response may be used to measure beyond the limits of the system and into the vascular system of the patient, by means of the needle/catheter. It is particularly important in the case of occlusions at the catheter, that one be able to ascertain the type of catheter used, based on its echo. In this way, the infusion system is able to recognize mistaken identifications of peridural and venous catheters, as well as the corresponding incorrect access points.

Electrical Monitoring:

With the electrical method, conductors are attached to the lines and other system elements, for conducting electrical signals. The conductors are attached in a way that ensures that an electrical connection is made when the elements are mechanically connected.

Depending on the complexity of the entire system, the individual elements of the system are provided with analogue and digital components. Thus, the elements of the system may be individually identified. If the individual elements are provided with analogue identification components (resistances, capacitances, inductivities), different statements may be made about the system, depending on the wiring.

If the components are electrically wired in series, the individual strands of the fluid system may be measured and in this way the entire system be recognized. If wired in parallel, the sum of the all of the connected elements may be calculated.

If the system is more complex, digital components, for example, microcontrollers, may be attached to elements of the system. This makes it possible to detect each individual element, including its position in the system. and also to recognize the individual operating states of the elements, such as valve settings, filter properties, etc. In this case, the power is supplied, for example, via a central controller that is attached to one or each of the infusion pumps in the system, via radio (for example, RFID) or induction. Each of the controllers attached to the elements has an identification number and one or more inputs that are used to read in information about the component, plus one or more outputs that are used to forward signals to additional controllers.

Optical Monitoring:

For particular applications, light may be used to recognize system connections. In this case, depending on the line and the fluid, light is sent either through the fluid in the tube line or through the material of the tube line. The evaluation is done analogously to the analysis of the acoustic measurement. It is possible in this way to recognize the connected elements and also to color code the lines.

Light may also be used to color-code different infusion strands, either for identifying the infused fluid or marking defective lines. The underlying assessment of a defective line may include a variety of conditions. For example, there may be a faulty connection in the line, or the maximal drip duration for an infusion tube connected to the patient has been reached. Internal illumination also may make it easier for a person to locate a line or a component that is to be identified or replaced.

The infusion system according to the invention enables the following advantages, which are described just very briefly:

By applying pattern recognition to the signals received at the sensor, it is possible to differentiate between information-carrying components of the signal and measurement errors, as well as artifacts. Thus, it is possible to recognize measurement errors caused by bends und sags in the line, coupling vibrations, 50 Hz signal drop-ins, pinching, as well as an inactive line.

The attenuation factor of an object, as well as the change in the wave form and phase of the signal caused by the object, may be used for identification purposes, when detecting the settings on T-connectors and other objects that are in or attached to the tube line.

Contact synchronization may be used when installing the system, as well as wireless synchronization methods, to synchronize the timing of the actuator/sensor modules.

Air bubbles do not have a negative influence on the functioning of the system.

With injection pumps, the pressure sensor/actuator may be integrated into the plunger that presses against the plunger pressure plate of the syringe (vibrating plate).

Peristaltic pumps send oscillations/sound signals based on their mechanics. The signals may be shaped into a clearly detectable form by modulating their velocities. The peristaltic elements may be used for signal detection or signal generation, also by means of special triggering. The ultrasound sensor for air detection may also be constructed to function as an actuator/sensor.

Roller pumps are a special type of peristaltic pumps. The rollers of these pumps may be modified to serve as the actuator.

Additional information may be applied/modulated onto the signals that are transmitted by the actuators; for example, type of medication and concentration, settings for the rate and pressure limits, pump ID, operating state including alarms, synchronization information including start and stop information. This is done by means of special signal wave forms, sequences, and signal characteristics, such as, for example, wave forms, frequencies (sweeps), pauses.

The properties of the tubes (length, inner diameter, wall diameter, E-Modulus, quality or nature of the tube) are determined by a change in the signal/transmission behavior over the length of the tube.

The temperature of the system components may be measured and used to compensate for changeable properties, such as signal line speed.

Phase shift of the signal and travel time may be used to measure flow rate. A correction for the different signal paths (through wall and fluid) may be used for this.

Parallel evaluations of multiple algorithms are compared and compiled to evaluate the signals.

The addition of new elements may be determined once by measuring and then be integrated into the model of the entire system.

It is understood that the embodiments described herein are merely illustrative of the present invention. Variations in the construction of the infusion system and method for monitoring the integrity of the infusion system may be contemplated by one skilled in the art without limiting the intended scope of the invention herein disclosed and as defined by the following claims.

What is claimed is:

1: A method of monitoring the integrity of an infusion system that has a plurality of system components that include an infusion source, a flow device, an infusion tube line, and a patient access point, the system administering a fluid to a patient, the method comprising the steps of:
a) providing a signal generator that generates a sensor signal and a sensor in the infusion system, wherein the sensor is placed a distance away from the signal generator;
b) providing an evaluation circuit that is operatively connected to the sensor;
c) transmitting the sensor signal through the fluid, from the signal generator to the sensor;
d) transmitting an input signal from the sensor to the evaluation circuit, the input signal carrying information that correlates with information carried by the sensor signal;
e) analyzing the input signal in the evaluation circuit to obtain and process the information carried by the sensor signal to obtain system information; and
f) providing a display means and displaying the system information on the display means.

2: The method of claim 1, wherein the sensor signal is a pressure signal.

3: The method of claim 1, wherein the sensor signal is a pulsed signal having one or more pulses.

4: The method of claim 1, wherein the sensor signal is modulated to carry a plurality of different signal frequencies.

5: The method of claim 1, further comprising the step of:
g) imprinting information on the sensor signal; and
h) transmitting the imprinted information from the sensor signal onto the input signal.

6: The method of claim 1, wherein a first actuator/sensor element is provided at a first location in the infusion system and a second actuator/sensor element provided at a second location, each actuator/sensor element having a signal generator and a sensor, the method further comprising the steps of:
i) emitting a first sensor signal from the first actuator/sensor element and a second signal from the second actuator/sensor element, so as to create a bi-directional signal transmission;
j) receiving the first sensor signal at the second actuator/sensor element and vice versa;
k) determining a travel time of the first sensor signal and the second sensor signal and comparing the travel time of the first sensor signal with the travel time of the second sensor signal; and
l) calculating a flow rate of the fluid through the infusion system, based on a difference in the travel time of two first and second sensor signals.

7: The method of claim 6, further comprising the step of:
m) automatically calculating a length of the infusion tube line, based on a travel time of the first and the second sensor signals.

8: The method of claim 6, further comprising the steps of:
n) providing control devices at two locations in the infusion system and actuating analysis of the sensor signal by simultaneously actuating the control devices.

9: The method of claim 1, the infusion system comprising a plurality of infusion tube lines and a plurality of signal generators and sensors, each signal generator transmitting a sensor signal through the infusion tube line, the method further comprising the steps of:
o) analyzing a travel time of each of the sensor signals;
p) determining positions and operating states of the system components in the infusion system;
q) determining complete setup and safety of the infusion system;
r) determining compatibility of the system components; and
s) graphically representing system information that includes flow rates of the fluids and the positions and operating states of the system components on the display monitor.

10: The method of claim 9, wherein the step of graphically representing the system information includes displaying the system information as a system map that displays information on the system components of the infusion system.

11: The method of claim 10, wherein displaying the system information as a system map includes the step of providing feedback that the infusion system is functioning properly or alternatively displaying an indication that there is an error in the infusion system.

12: The method of claim 1, wherein the system components further include one or more stopcocks, multi-port valves, filters, and branching connectors.

13: The method of claim 1, wherein the sensor signal is modulated and carries information relating to a wireless component, the method further comprising the step of:
t) analyzing the sensor signal for the information that identifies the wireless component.

14: The method of claim 1 further comprising the step of:
u) automatically transmitting information on the type of the fluid contained in the infusion source, medication prescription information, and pharmacological information to the evaluation circuit for processing and incorporation into system information.

15: The method of claim 1, further comprising the steps of:
v) receiving an echo of the sensor signal by the sensor;
w) measuring the echo to determine a type and location of a component causing the echo; and
x) evaluating the signal echo to determine a source that is causing a restriction of the flow rate.

16: The method of claim 1, further comprising the steps of:
y) analyzing the transmission behavior of the system components in the infusion system; and
z) virtually simulating a second infusion system and presenting information as to properties and functioning of the simulated system.

17: The method of claim 1 further comprising the step of:
aa) analyzing an echo of a transmitted sensor signal to determine the type of the patient access point that is connected to a patient.

18: The method of claim 1, wherein the system information includes information on flow rate of the fluid, including information on any abnormality in flow rate.

19: The method of claim 1, wherein the abnormality in flow rate is caused by a blockage and/or a leakage.

20: The method of claim 1, further comprising the step of detecting a presence of gas bubbles in the fluid.
